(19) United States
(12) Patent Application Publication
(10) Pub. No.: US 2021/0004573 A1
(43) Pub. Date: Jan. 7, 2021

(54) SENTIMENT ANALYSIS

(71) Applicant: HEWLETT-PACKARD DEVELOPMENT COMPANY, L.P., SPRING, TX (US)

(72) Inventors: MOHIT GUPTA, SAN DIEGO, CA (US); LUCAS PETTIT, SAN DIEGO, CA (US); CHRIS KRUGER, SAN DIEGO, CA (US)

(21) Appl. No.: 16/763,494

(22) PCT Filed: Mar. 8, 2018
(86) PCT No.: PCT/US18/21513

§ 371 Date: May 12, 2020

(51) Int. Cl. G06K 9/00; G08B 21/18

(52) U.S. Cl. CPC G06K 9/00302; G06K 9/00248; G08B 21/18; G06K 9/00281; G06K 9/00288

(57) ABSTRACT

Example implementations relate to sentiment analysis. A computing device may comprise a processing resource, and a memory resource storing machine-readable instructions to cause the processing resource to receive a digital image of a subject, analyze the digital image to detect facial features of the subject, analyze the detected facial features to determine an identity of the subject, determine a sentiment level of the subject using a sentiment analysis, where the sentiment analysis uses the detected facial features and the identity of the subject to determine the sentiment level of the subject, and display the sentiment level of the subject via a display.

### BACKGROUND

Identifying the sentiment of customers and employees can be a factor in providing services. Subjects, such as customers and/or employees can be surveyed before, during, and/or after a transaction by asking if the transaction experience was satisfactory or not. Customers can be surveyed post-transaction based on their recollection of an event, time, and/or day of the transaction.

### BRIEF DESCRIPTION OF THE DRAWINGS

FIG. 1 is a diagram of an example system to perform sentiment analysis according to the disclosure.

FIG. 2 is a block diagram of an example of a computing device to perform sentiment analysis according to the disclosure.

FIG. 3 is a block diagram of an example of a system consistent with the disclosure.

FIG. 4 is an example of a computing device to perform sentiment analysis according to the disclosure.

### DETAILED DESCRIPTION

Surveys, reviews, and/or voice detection of subjects before, during, and/or after a transaction to determine a sentiment level of the subjects can allow for insight into trends and early signs of issues. However, the analysis of surveys, reviews, and/or voice detection can be limited to a subgroup of subjects who are either happy or upset enough to want to leave a review, ask for customer assistance, and/or desire to take part in a survey. Further, surveys can be time-consuming to create and may be subject to bias in question phrasing, reviews can be fraudulent, analysis is typically gathered post-transaction and may be dependent on a subject's recollection of the transaction, and surveys and reviews may be subject to the bias of the creator of the surveys and reviews.

Sentiment analysis, according to the disclosure, can allow for a subject's sentiment level to be determined and monitored. For example, the subject can be subjected to sentiment analysis while they are monitored by a camera. As used herein, the term “subject” can, for example, refer to a person as an object of interest. Sentiment analysis can provide for insights into a subject's sentiment regarding a transaction while removing the workload of creating and filling out surveys and/or reviews and deriving meaning from those surveys and/or reviews.

Sentiment analysis, according to the disclosure, can refer to determining an attitude of a speaker, writer, or other subject with respect to some topic or the overall contextual polarity or emotional reaction to a document, interaction, or event. As used herein, the term “sentiment level” can, for example, refer to a degree to which a subject has a sentiment. Sentiment levels can include a happy sentiment level, a frustrated sentiment level, an upset sentiment level, and/or a satisfied sentiment level, among other types of sentiment levels.

Determining a sentiment level of a subject may include analyzing a subject's sentiment level using the subject's identity based on facial features. In some instances, facial features may be determined via a digital image of the subject received from a camera. In some instances, facial features may include an element of the face. As used herein, an “element of a face” can, for example, refer to an ear, nose, mouth, hair, jaw, and/or cheekbones of a subject, among other types of facial elements of a subject.

Sentiment analysis via sentiment level determination according to the disclosure can allow for analyzing and determining an identity of a subject from facial features. As used herein, the term, “identity”, can for example refer to a distinguishing character or personality of an individual. A subject's identity can distinguish the subject from other subjects. A sentiment level can be determined for each subject, where the subjects are distinguishable via their respective identities.

A sentiment level may be displayed based on the determination of a sentiment level of a subject via sentiment analysis. A subject's sentiment level, identity, as well as contextual data may be stored for future use to improve customer satisfaction. In some examples, an alert in response to the determined sentiment level being different from a previous sentiment level may be generated, as is further described herein.

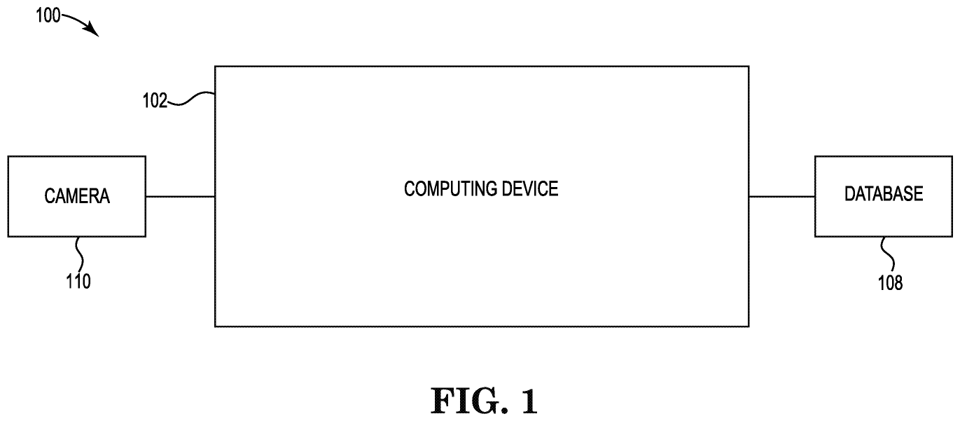

FIG. 1 is a diagram of an example system 100 to perform sentiment analysis according to the disclosure. As illustrated in FIG. 1, the system 100 may include a computing device 102, a camera 110, and a database 108.

System 100 may include database 108. Database 108 can perform functions related to sentiment analysis. In some examples, database 108 can be included in computing device 102. In some instances, database 108 can be located remote from computing device 102 as illustrated in FIG. 1.

Data can be transmitted to and/or from database 108 via computing device 102 via a network relationship. For example, data can be transmitted to and/or from database 108 via computing device 102 via a wired or wireless network. As used herein, “data” can refer to a set of values of qualitative or quantitative variables. The data included in database 108 can be hardware data and/or software data of the database 108, among other types of data.

The wired or wireless network can be a network relationship that connects the database 108 to computing device 102. Examples of such a network relationship can include a local area network (LAN), wide area network (WAN), personal area network (PAN), a distributed computing environment (e.g., a cloud computing environment), storage area network (SAN), Metropolitan area network (MAN), a cellular communications network, and/or the Internet, among other types of network relationships.

Computing device 102 can receive from camera 110 a digital image of a subject. As used herein, a “camera” can refer to a device for recording visual images in the form of photographs, film, and/or video signals, such as, for example, compact digital cameras such as Digital Single Lens Reflex (DSLR) Cameras, mirrorless cameras, infrared (IR) cameras, action cameras, 360 cameras, and/or cameras, among other types of cameras.

Digital images may be periodically transmitted to computing device 102. In some examples, camera 110 may transmit digital images to computing device 102 based on a predetermined time period. For example, computing device 102 can transmit a digital image to computing device 102 every fifteen minutes, every ten minutes, and/or any other time period.

In some examples, camera 110 may transmit digital images to computing device 102 in response to a subject's change in position. For example, in response to a subject changing position, camera 110 can take and transmit a digital image to computing device 102. A change in position can, for instance, refer to a change in physical position of the subject. For example, the subject may move their arm, take a step to a different physical location than where the subject was previously standing, may move their torso, among other types of changes in position of a subject.

In some examples, camera 110 may transmit digital images to computing device 102 in response to an action by a subject causing the camera 110 to transmit digital images to computing device 102. For instance, camera 110 may take a digital image upon a new client's arrival and transmit the digital image to computing device 102. In some examples, the action by the subject can include picking up a predetermined product, standing or entering a predetermined area, etc.

Computing device 102 can analyze the digital image received from camera 110 to detect facial features of a subject. As used herein, the term “facial feature” can for example, refer to a distinguishing element of a face. For example, element of the subject's face may be an ear, nose, mouth, hair, jaw, and/or cheekbones of the subject.

Facial features can be detected by computing device 102 via object detection. Object detection can, for example, refer to detecting instances of semantic objects in digital images. Semantic objects can include facial features. For example, computing device 102 can utilize object detection to detect facial elements such as a subject's ear, nose, eye, mouth, hair, jaw, and/or cheekbones, among other facial features and/or combinations thereof.

Computing device 102 can receive the digital image of the subject from camera 110 and analyze the detected facial features of the subject received from camera 110. Analyzing the detected facial features can include analyzing an element of the subject's face. For example, computing device 110 can analyze an ear, nose, eye, mouth, hair, jaw, cheekbones, and/or combinations thereof of the subject's face.

Analyzing an element of the subject's face can include determining various characteristics about the element of the subject's face. For example, characteristics of an element of the subject's face can include a shape of the element, a size of the element, a color of the element, distinguishing features of the element, among other types of characteristics. For example, computing device 102 may analyze an element of the subject's face such as the subject's eye. Analyzing the subject's eye may include determining a shape of the eye, size of the eye, color of the eye, etc.

Computing device 102 can analyze the detected facial features to determine an identity of a subject. In some examples, computing device 102 can identify a subject as an existing subject or as a new subject, as is further described herein.

In some examples, computing device 102 can identify the subject as an existing subject. For example, computing device 102 may receive a digital image from camera 110. Computing device 102 may then analyze the detected facial features of the subject based on an element of a subject's face. For example, computing device 102 may analyze a subject's nose, mouth, and jaw. Based on the analysis, computing device 102 may identify the subject as an existing subject. For instance, if facial features included in the image received from camera 110 match the facial features of an existing image included in database 108, computing device 102 can identify the subject as an existing subject.

In some examples, computing device 102 can identify the subject as a new subject. For example, computing device 102 may receive a digital image from camera 110. Computing device 102 may then analyze the detected facial features of the subject based on an element of a subject's face. For example, computing device 102 may analyze a subject's nose, mouth, and jaw. Based on the analysis, computing device 102 may not identify the subject as an existing subject. For instance, if facial features included in the image received from camera 110 do not match the facial features of an existing image included in database 108, computing device 102 can identify the subject as a new subject.

Although computing device 102 is described above as utilizing a subject's nose, mouth, and jaw, examples of the disclosure are not so limited. For example, computing device 102 can utilize a subject's ear(s), nose, mouth, hair, jaw, and/or cheekbones, and/or any other facial element and/or combination thereof to determine the identity of a subject.

Computing device 102 can determine a sentiment level of a subject using a sentiment analysis. For example, computing device 102 can determine the sentiment level by detecting facial features and the identity of a subject. Computing device 102 can determine a sentiment analysis via machine learning. For example, computing device 102 can utilize decision tree learning, artificial neural networks, deep learning, inductive logic programming, support vector machines, Bayesian networks, and/or learning classifier systems to determine a sentiment analysis, among other types of machine learning techniques.

In some examples, computing device 102 can determine the subject's sentiment level based on a facial expression of the subject. For example, computing device 102 may determine a subject's sentiment levels as a happy sentiment level, a frustrated sentiment level, an upset sentiment level, and/or a satisfied sentiment level, among other types of sentiment levels. For example, computing device 102 can determine the subject's sentiment level as happy based on the mouth of the subject being oriented in a smile. In some examples, computing device 102 can determine the subject's sentiment level as upset based on the subject's eyebrows being turned down.

In some examples, computing device 102 to can determine a subject's sentiment level based on an identity of the subject. For example, if computing device 102 identifies a subject as an existing subject, computing device 102 can determine the sentiment level to be the previous sentiment level of the existing subject. Further, based on the analysis of the subject, computing device 102 can update the subject's sentiment level by comparing the subject's sentiment level with facial features and related sentiment levels of subjects, received from database 108.

Computing device 102 can determine customer satisfaction based on the determined sentiment level of the subject. As used herein, the term “customer satisfaction” can, for example, refer to a measure of how a product or service meets a customer expectation. For example, computing device 102 can determine the customer satisfaction utilizing the facial features analysis, as is further described herein.

In some examples, computing device 102 may determine a customer satisfaction level as dissatisfied based on the determined sentiment level. For example, computing device 102 can identify a subject as a new customer and determine the sentiment level of the subject based on facial features analysis. For example, the sentiment level may be determined by computing device 102 as frustrated. Based on the determination of the subject's sentiment level as frustrated, computing device 102 may determine the subject has a dissatisfied customer satisfaction level.

In some examples, computing device 102 may identify a subject as an existing customer based on facial features analysis. Computing device 102 may then determine the subject's sentiment level as a happy sentiment level by comparing the subject's facial features with sentiment levels stored in and received from database 108. Based on the determination of subject's sentiment level as a happy sentiment level, computing device 102 may determine the customer is satisfied.

Sentiment level information stored in database 108 can include existing subjects' information. Sentiment level information stored in database 108 can be information from other subjects, collected in various places and at various points in time.

Computing device 102 can display the sentiment level of the subject via a display. As used herein, the term “display” can, for example, refer to an output device which can display information via a screen. A display may include a television, computer monitor, mobile device display, other type of display device, or any combination thereof, which can receive and output a video signal. The display can be a liquid crystal display (LCD), LED display, organic light-emitting diode (OLED) display, polymer light-emitting diode (PLED) display, micro-LED display, electronic paper display (EPD), bi-stable display, and/or a quantum-dot LED (QLED) display, among other types of displays.

In some examples, computing device 102 may identify determine a subject's sentiment level and display the sentiment level via a display. In one example, an existing subject's sentiment level may be determined as “dissatisfied”. The dissatisfied sentiment level may be displayed on a display so that an employee, supervisor, and/or other user may view the determined sentiment level. Based on the determined sentiment level, appropriate action can be taken. In some examples, further employee training can be performed to improve customer sentiment levels and customer satisfaction. In some examples, the subject may be given coupons or other discounts in order to improve customer sentiment levels. In some examples, appropriate personnel may be notified based on the subject's sentiment level.

Computing device 102 can generate a report including the determined sentiment level and/or the past sentiment level of the subject. As used herein, the term “report” can, for example, refer to an account or statement describing an event. For example, the report generated by computing device 102 can include a sentiment level of the subject, including, for instance, whether the subject has a happy, frustrated, upset, and/or a dissatisfied sentiment level, among other types of sentiment levels, whether the subject is a new or existing subject, the customer satisfaction of the subject, including, for instance, whether the subject is satisfied, dissatisfied, among other types of satisfaction levels, etc.

The report can include information to allow personnel, such as a supervisor and/or employee, to determine whether to take action to improve the subject's experience by improving their sentiment level and/or customer satisfaction, to give an employee further training, etc. In some examples, the report can be displayed via a display. In some examples, the report can be printed by an imaging device, such as a printer, such that the report can be physically distributed among personnel.

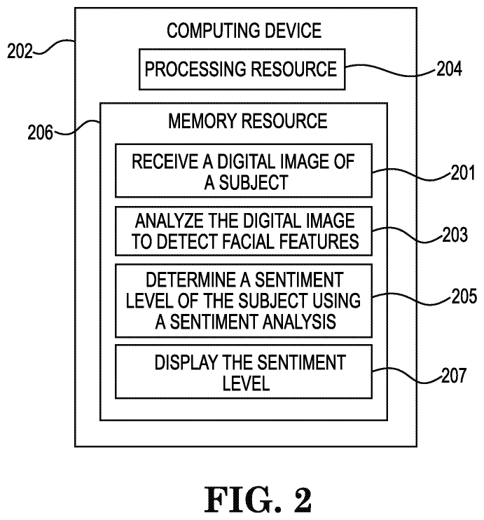

FIG. 2 is a block diagram 220 of an example computing device 202 for sentiment analysis consistent with the disclosure. As described herein, the computing device 202 may perform a number of functions related to sentiment analysis. Although not illustrated in FIG. 2, the computing device 202 may include a processor and a machine-readable storage medium. Although the following descriptions refer to a single processor and a single machine-readable storage medium, the descriptions may also apply to a system with multiple processors and multiple machine-readable storage mediums. In such examples, the computing device 202 may be distributed across multiple machine-readable storage mediums and the computing device 202 may be distributed across multiple processors. Put another way, the instructions executed by the computing device 202 may be stored across multiple machine-readable storage mediums and executed across multiple processors, such as in a distributed or virtual computing environment.

Processing resource 204 may be a central processing unit (CPU), a semiconductor based microprocessor, and/or other hardware devices suitable for retrieval and execution of machine-readable instructions 201, 203, 205, 207, stored in memory resource 206. Processing resource 204 may fetch, decode, and execute instructions 201, 203, 205, 207. As an alternative or in addition to retrieving and executing instructions 201, 203, 205, 207, processing resource 204 may include a plurality of electronic circuits that include electronic components for performing the functionality of instructions 201, 203, 205, 207.

Memory resource 206 may be any electronic, magnetic, optical, or other physical storage device that stores executable instructions 201, 203, 205, 207 and/or data. Thus, memory resource 206 may be, for example, Random Access Memory (RAM), an Electrically-Erasable Programmable Read-Only Memory (EEPROM), a storage drive, an optical disc, and the like. Memory resource 206 may be disposed within computing device 202, as shown in FIG. 2. Additionally, and/or alternatively, memory resource 206 may be a portable, external or remote storage medium, for example, that allows computing device 202 to download the instructions 201, 203, 205, 207 from a portable/external/remote storage medium.

Computing device 202 may include instructions 201 stored in the memory resource 206 and executable by the processing resource 204 to receive a digital image of a subject. For example, computing device 202 may execute instructions 201 via the processing resource 204 to receive, from a camera, a digital image of a subject.

For example, digital images taken by a camera may be periodically transmitted to computing device 202. In one example a camera may transmit digital images to computer device 202 periodically, such as every fifteen minutes, and/or in response to a subject's change in position.

In one example, a camera may transmit digital images to computing device 202 in response to a subject's triggered action. For instance, an employee may trigger a camera to take a digital image upon a new subject's arrival, and transmit the digital image to computing device 202.

Computing device 202 may include instructions 203 stored in the memory resource 206 and executable by the processing resource 204 to analyze the digital image to detect facial features. For example, computing device 202 may execute instructions 203 via the processing resource 204 to analyze the digital image to detect facial features of the subject. Facial elements of the subject can, for example, include an ear nose, mouth, eye, hair, jaw, and/or cheekbones of the subject, among other facial elements and/or combinations thereof.

Computing device 202 may include instructions 205 stored in the memory resource 206 and executable by the processing resource 204 to determine a sentiment level of the subject using a sentiment analysis. For example, computing device 202 may execute instructions 205 via the processing resource 204 to determine a sentiment level of the subject using a sentiment analysis, where the sentiment analysis uses the detected facial features and the identity of the subject to determine the sentiment level of the subject.

In one example, computing device 202 can determine the identity of a subject as an existing subject. In some examples, computing device 202 can determine the identity of the subject as a new subject.

In some examples, computing device 202 can analyze the detected facial features of a subject based on an element of the subject's face. For example, the computing device 202 can to analyze a subject's nose, mouth, and jaw, Computing device 202 can compare the facial features from the digital image with facial features of existing images in a database. In response to the facial features from the digital image matching facial features of an existing image in the database, computing device 202 can identify the subject as an existing subject.

In some examples, computing device 202 can analyze the detected facial features of a subject based on an element of the subject's face. For example, the computing device 202 can to analyze a subject's nose, mouth, and jaw. Computing device 202 can compare the facial features from the digital image with facial features of existing images in a database. In response to the facial features from the digital image not matching facial features of an existing image in the database, computing device 202 can identify the subject as a new subject.

Computing device 202 may include instructions 207 stored in the memory resource 206 and executable by the processing resource 204 to display the sentiment level. For example, computing device 202 may execute instructions 207 via the processing resource 204 to display the sentiment level via a display.

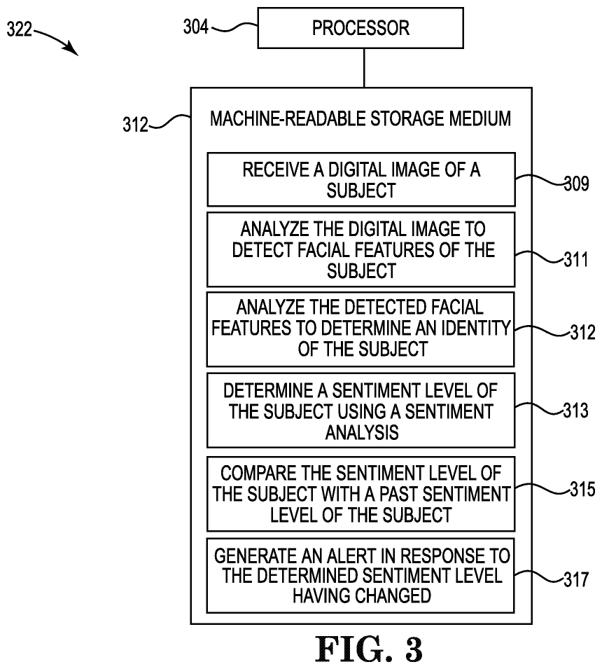

FIG. 3 is a block diagram of an example of a system 322 consistent with the disclosure. In the example of FIG. 3, system 322 includes a processor 304 and a machine-readable storage medium 312. Although the following descriptions refer to an individual processing resource and an individual machine-readable storage medium, the descriptions may also apply to a system with multiple processing resources and multiple machine-readable storage mediums. In such examples, the instructions may be distributed across multiple machine-readable storage mediums and the instructions may be distributed across multiple processing resources. Put another way, the instructions may be stored across multiple machine-readable storage mediums and executed across multiple processing resources, such as in a distributed computing environment.

Processor 304 may be a central processing unit (CPU), microprocessor, and/or other hardware device suitable for retrieval and execution of instructions stored in machine-readable storage medium 312. In the particular example shown in FIG. 3, processor 304 may receive, determine, and send instructions 309, 311, 313, 315, 317. As an alternative or in addition to retrieving and executing instructions, processor 304 may include an electronic circuit comprising a number of electronic components for performing the operations of the instructions in machine-readable storage medium 312. With respect to the executable instruction representations or boxes described and shown herein, it should be understood that part or all of the executable instructions and/or electronic circuits included within one box may be included in a different box shown in the figures or in a different box not shown.

Machine-readable storage medium 312 may be any electronic, magnetic, optical, or other physical storage device that stores executable instructions. Thus, machine-readable storage medium 312 may be, for example, Random Access Memory (RAM), an Electrically-Erasable Programmable Read-Only Memory (EEPROM), a storage drive, an optical disc, and the like. The executable instructions may be “installed” on the system 322 illustrated in FIG. 3. Machine-readable storage medium 312 may be a portable, external or remote storage medium, for example, that allows the system 322 to download the instructions from the portable/external/remote storage medium. In this situation, the executable instructions may be part of an “installation package”. As described herein, machine-readable storage medium 312 may be encoded with executable instructions for sentiment analysis.

Instructions 309 to receive a digital image, when executed by processor 304, may cause system 322 to receive a digital image of a subject. For example, a computing device including processor 304 and machine-readable storage medium 312 can receive a digital image of a subject from a camera.

Instructions 311 to analyze the digital image to detect facial features of the subject, when executed by processor 304, may cause system 322 to analyze the digital image to detect facial features of the subject. Facial features of the subject can include an element of the subject's face. For example, elements of a subject's face may include an ear, nose, mouth, hair, jaw, and/or cheekbones of the subject, among other facial elements and/or combinations thereof.

Instructions 312 to analyze the detected facial features to determine an identity of the subject, when executed by processor 304, may cause system 322 to analyze the detected facial features to determine an identity of the subject by comparing the detected facial features to facial features included in a database. In some examples, the computing device can determine the subject to be an existing subject in response to the detected facial features matching the facial features included in the database. In some examples, the computing device can determine the subject to be a new subject in response to the detected facial features not matching the facial features included in the database.

Instructions 313 to determine a sentiment level of the subject using a sentiment analysis, when executed by processor 304, may cause system 322 to determine a sentiment level of the subject using a sentiment analysis, where the sentiment analysis uses the detected facial features and an identity of the subject to determine the sentiment level of the subject.

Instructions 315 to compare the sentiment level of the subject with a past sentiment level of the subject, when executed by processor 304, may cause system 322 to compare the sentiment level of the subject with a past sentiment level of the subject in response to the subject being an existing subject.

Instructions 317 to generate an alert in response to the determined sentiment level having changed, when executed by processor 304, may cause system 322 to generate an alert in response to the determined sentiment level being changed from the past sentiment level. For example, an alert may be generated such that an employee can be notified that a sentiment level of the subject has changed. In some examples, the employee can, in response to the sentiment level having changed, approach the subject differently, offer the subject coupons, and/or other actions.

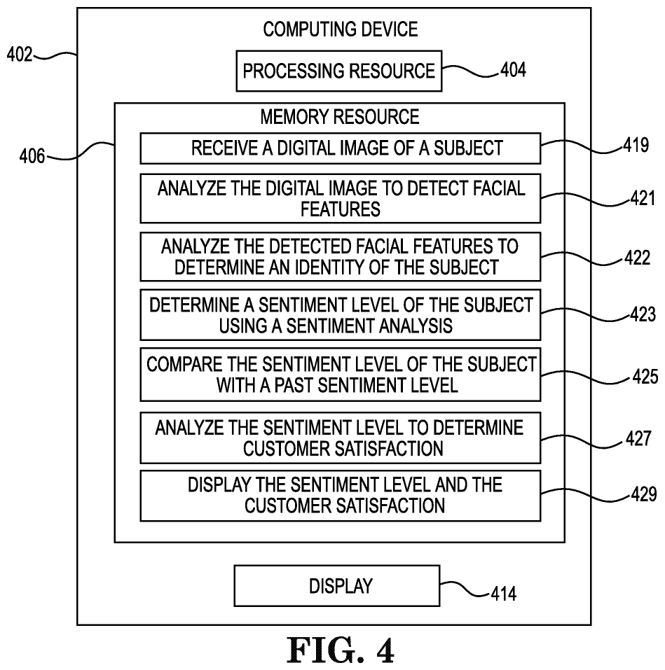

FIG. 4 is a block diagram of an example computing device 402 to perform sentiment analysis consistent with the disclosure. As described herein, the computing device 402 may perform a number of functions related to sentiment analysis.

Processing resource 404 may be a central processing unit (CPU), a semiconductor based microprocessor, and/or other hardware devices suitable for retrieval and execution of machine-readable instructions 419, 421, 423, 425, 427, 429 stored in memory resource 406. Processing resource 404 may fetch, decode, and execute instructions 419, 421, 423, 425, 427, 429. As an alternative or in addition to retrieving and executing instructions 419, 421, 423, 425, 427, 429, processing resource 404 may include a plurality of electronic circuits that include electronic components for performing the functionality of instructions 419, 421, 423, 425, 427, 429.

Memory resource 406 may be any electronic, magnetic, optical, or other physical storage device that stores executable instructions 419, 421, 423, 425, 427, 429 and/or data. Thus, memory resource 406 may be, for example, Random Access Memory (RAM), an Electrically-Erasable Programmable Read-Only Memory (EEPROM), a storage drive, an optical disc, and the like. Memory resource 406 may be disposed within computing device 402, as shown in FIG. 4. Additionally, and/or alternatively, memory resource 406 may be a portable, external or remote storage medium, for example, that allows computing device 402 to download the instructions 419, 421, 423, 425, 427, 429 from a portable/external/remote storage medium.

Computing device 402 may include instructions 419 stored in the memory resource 406 and executable by the processing resource 404 to receive a digital image of a subject. For example, computing device 402 may execute instructions 419 via the processing resource 404 to receive, from a camera, a digital image of a subject.

Computing device 402 may include instructions 421 stored in the memory resource 406 and executable by the processing resource 404 to analyze the digital image to detect facial features. For example, computing device 402 may execute instructions 421 via the processing resource 404 to analyze the digital image to detect facial features of the subject. Facial elements of the subject can, for example, include an ear nose, mouth, eye, hair, jaw, and/or cheekbones of the subject, among other facial elements and/or combinations thereof.

Computing device 402 may include instructions 422 stored in the memory resource 406 and executable by the processing resource 404 to analyze the detected facial features to determine an identity of the subject. For example, computing device 402 may execute instructions 422 via the processing resource 404 to analyze the detected facial features to determine an identity of the subject by comparing the detected facial features to facial features included in a database. The subject can be determined to be an existing subject in response to the detected facial features matching the facial features included in the database. The subject can be determined to be a new subject in response to the detected facial features not matching the facial features included in the database.

Computing device 402 may include instructions 423 stored in the memory resource 406 and executable by the processing resource 404 to determine a sentiment level of the subject using a sentiment analysis. For example, computing device 402 may execute instructions 423 via the processing resource 404 to determine a sentiment level of the subject using a sentiment analysis, where the sentiment analysis uses the detected facial features and the identity of the subject to determine the sentiment level of the subject.

Computing device 402 may include instructions 425 stored in the memory resource 406 and executable by the processing resource 404 to compare the sentiment level of the subject with a past sentiment level. For example, computing device 402 may execute instructions 425 via the processing resource 404 to compare the sentiment level of the subject with a past sentiment level of the subject in response to the subject being an existing subject.

Computing device 402 may include instructions 427 stored in the memory resource 406 and executable by the processing resource 404 to analyze the sentiment level to determine customer satisfaction. For example, the customer may be satisfied or dissatisfied.

Computing device 402 may include instructions 429 stored in the memory resource 406 and executable by the processing resource 404 to display the sentiment level and the customer satisfaction. For example, computing device 402 may execute instructions 429 via the processing resource 404 to display the sentiment level and the customer satisfaction of the subject via display 414.

Display 414 can be, for instance, a touch-screen display. As previously described in connection with FIG. 1, the display may include a television, computer monitor, mobile device display, other type of display device, or any combination thereof, which can receive and output a video signal.

The figures herein follow a numbering convention in which the first digit corresponds to the drawing figure number and the remaining digits identify an element or component in the drawing. Similar elements or components between different figures may be identified by the use of similar digits. For example, 102 may reference element “02” in FIG. 1, and a similar element may be referenced as 202 in FIG. 2. Elements shown in the various figures herein can be added, exchanged, and/or eliminated so as to provide a plurality of additional examples of the disclosure. In addition, the proportion and the relative scale of the elements provided in the figures are intended to illustrate the examples of the disclosure and should not be taken in a limiting sense. Further, as used herein, “a plurality of” an element and/or feature can refer to more than one of such elements and/or features.

What is claimed:

1. A computing device, comprising:
a processing resource; and
a memory resource storing machine readable instructions to cause the processing resource to:
receive, from a camera, a digital image of a subject;
analyze the digital image to detect facial features of the subject;
analyze the detected facial features to determine an identity of the subject;
determine a sentiment level of the subject using a sentiment analysis, wherein the sentiment analysis uses the detected facial features and the identity of the subject to determine the sentiment level of the subject; and
display the sentiment level of the subject via a display.

2. The computing device of claim 1, wherein the processing resource executes the machine readable instructions to analyze the facial features by detecting an element of the subject's face.

3. The computing device of claim 2, wherein the element of the subject's face is to be selected from a group consisting of an ear, nose, mouth, hair, jaw, and cheekbones of the subject.

4. The computing device of claim 1, wherein the processing resource executes the machine readable instructions to identify the subject as an existing subject or a new subject based on the subject's facial features.

5. The computing device of claim 1, wherein the processing resource executes the machine readable instructions to determine the subject's sentiment level based on a facial expression of the subject.

6. The computing device of claim 1, wherein the processing resource executes the machine readable instructions to determine the subjects' sentiment level based on an identity of the subject.

7. The computing device of claim 1, wherein the processing resource executes the machine readable instructions to determine a customer satisfaction based on the determined sentiment level of the subject.

8. A non-transitory machine-readable medium storing instructions executable by a processing resource to:
receive, from a camera, a digital image of a subject;
analyze the digital image to detect facial features of the subject;
analyze the detected facial features to determine an identity of the subject by comparing the detected facial features to facial features included in a database, wherein:
the subject is to be determined to be an existing subject in response to the detected facial features matching the facial features included in the database;
the subject is to be determined to be a new subject in response to the detected facial features not matching the facial features included in the database;
determine a sentiment level of the subject using a sentiment analysis, wherein the sentiment analysis uses the detected facial features and the identity of the subject to determine the sentiment level of the subject;
compare, in response to the subject being an existing subject, the sentiment level of the subject with a past sentiment level of the subject; and
generate an alert in response to the determined sentiment level having changed from the past sentiment level.

9. The medium of claim 8, comprising instructions to analyze the facial features of the subject from an element of the subject's face to identify the subject as a new subject or an existing subject.

10. The medium of claim 9, wherein the existing subject is an employee, and wherein the instructions are executable the processing resource to generate an alert in response to the determined sentiment level having changed from the past sentiment level.

11. The medium of claim 9, wherein the existing subject is a returning customer, and wherein the instructions are executable by the processing resource to generate an alert in response to the determined sentiment level being worse than the past sentiment level.

12. The medium of claim 8, comprising instructions to generate a report including the determined sentiment level and the past sentiment level.

13. A computing device, comprising:
a display;
a processing resource; and
a memory resource storing machine readable instructions to cause the processing resource to:
receive, from a camera, a digital image of a subject;
analyze the digital image to detect facial features of the subject;
analyze the detected facial features to determine an identity of the subject by comparing the detected facial features to facial features included in a database, wherein:
the subject is determined to be an existing subject in response to the detected facial features matching the facial features included in the database;
the subject is determined to be a new subject in response to the detected facial features not matching the facial features included in the database;
determine a sentiment level of the subject using a sentiment analysis;
compare, in response to the subject being an existing subject, the sentiment level of the subject with a past sentiment level of the subject;
analyze the sentiment level to determine customer satisfaction; and
display the sentiment level and the customer satisfaction of the subject via the display.

14. The computing device of claim 13, wherein the sentiment level is to be determined from a group consisting of a happy sentiment level, a frustrated sentiment level, an upset sentiment level, and a satisfied sentiment level.

15. The computing device of claim 13, wherein the subjects' sentiment level is to be analyzed from contextual information including a date, a time, a duration of a visit, a location of the workstation, and combinations thereof.
